(19) United States
(12) Patent Application Publication
(10) Pub. No.: US 2011/0023139 A1
(43) Pub. Date: Jan. 27, 2011

(54) GENOMIC EDITING OF GENES INVOLVED IN CARDIOVASCULAR DISEASE

(75) Inventors: Edward Weinstein, St. Louis, MO (US); Xiaoxia Cui, St. Louis, MO (US); Phil Simmons, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,198

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. A61K 49/00; A01K 67/027; C12N 5/10; C12N 9/16; C07H 21/04; A61P 43/00

(52) U.S. Cl. 800/3; 800/13; 800/14; 800/15; 800/16; 800/17; 800/18; 435/325; 435/196; 435/350; 435/351; 435/352; 435/353; 435/363; 536/23.1; 536/23.2

(57) ABSTRACT

The present invention provides genetically modified animals and cells comprising edited chromosomal involved in cardiovascular disease. In particular, the animals or cells are generated using a zinc finger nuclease-mediated editing process. The invention also provides zinc finger nucleases that target chromosomal sequences involved in cardiovascular disease and the nucleic acids encoding said zinc finger nucleases. Also provided are methods of using the genetically modified animals or cells disclosed herein to screen agents for toxicity and other effects.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified animals or cells comprising at least one edited chromosomal sequence involved in cardiovascular disease. In particular, the invention relates to the use of a zinc finger nuclease-mediated disruption process to edit chromosomal sequences involved in cardiovascular disease.

### BACKGROUND OF THE INVENTION

Cardiovascular disease is any disease which affects the cardiovascular system, specifically involving the heart and blood vessels. Almost 1 million Americans die of cardiovascular disease a year, specifically from heart disease and stroke. Early medical detection of cardiovascular disease is imperative to preventing death, however, early detection is difficult since there is not a good animal model available for cardiovascular disease. Further, it is difficult to monitor the affect of pharmaceuticals and other treatments for cardiovascular disease.

The vast majority of drugs (approximately 91%) fail to successfully proceed through the three phases of drug testing in humans. A majority of those drugs that fail do so because of unforeseen toxicology in human patients despite the fact that all of these drugs had been tested in animal models and were found to be safe. This is because toxicology testing is performed in animals, and animal proteins differ from the orthologous proteins in humans.

It has been found that the genes Cacna1C, Sod1, Pten, Ppar (alpha), Apo E, and Leptin are involved in cardiovascular diseases. Calcium channel, voltage-dependent, L type, alpha 1C subunit is a subunit of L-type voltage dependent calcium channel encoded by the Cacna1C gene. Voltage-gated calcium channels (CaV) are present in the membrane of most excitable cells and mediate calcium influx in response to depolarization. They regulate intracellular processes such as contraction, secretion, neurotransmission and gene expression. Sod1 (superoxide dismutase [Cu—Zn]) is an enzyme that in humans is encoded by the Sod1 gene. Sod1 binds copper and zinc ions and is one of three isozymes responsible for destroying free superoxide radicals in the body. Pten (phosphatase and tensin homolog) is a protein that, in humans, is encoded by the Pten gene. Pten acts as a tumor suppressor gene through the action of its phosphatase protein product, which is involved in the regulation of the cell cycle, preventing cells from growing and dividing too rapidly. Ppar(alpha) (peroxisome proliferator-activated receptor alpha) is a nuclear receptor protein encoded by the PPARA gene. Peroxisome proliferators receptor ligands induce an increase in the size and number of peroxisomes. Perioxisomes are subcellular organelles found in plants and animals that contain enzymes for respiration and for cholesterol and lipid metabolism.

What is needed are animal models in which these genes involved in cardiovascular disease are mutated, including gene knockouts, multiple mutant lines, and/or expression of over-expression of alleles that either cause disease or are associated with disease in humans. Further, what is needed is a way to create these animal models without the time investment and research hurdles facing traditional technology creating knock-out mice. Additionally, a model for cardiovascular disease which is closer to the human genome than a mouse model is also desired.

### SUMMARY OF THE INVENTION

One aspect of the present disclosure encompasses a genetically modified animal comprising at least one edited chromosomal sequence involved in cardiovascular disease.

A further aspect provides a non-human embryo comprising at least one RNA molecule encoding a zinc finger nuclease that recognizes a chromosomal sequence encoding a protein involved in cardiovascular disease, and, optionally, at least one donor polynucleotide comprising a sequence encoding an ortholog of the protein involved in cardiovascular disease.

Another aspect provides for a genetically modified cell comprising at least one edited chromosomal sequence involved in cardiovascular disease.

An alternate aspect provides a zinc finger nuclease comprising (a) a zinc finger DNA binding domain that binds a sequence having at least about 80% sequence identity with a sequence chosen from SEQ ID NOs:4, 5, 6 and 7, and (b) a cleavage domain.

Another aspect provides a nucleic acid sequence that is recognized by a zinc finger nuclease. The nucleic acid sequence has at least about 80% sequence identity with a sequence chosen from SEQ ID NOs: 4, 5, 6 and 7.

Yet another aspect encompasses a method for assessing the effect of an agent in an animal. The method comprises contacting a genetically modified animal comprising at least one edited chromosomal sequence involved in cardiovascular disease with the agent, and comparing results of a selected parameter to results obtained from contacting a wild-type animal with the same agent. The selected parameter is chosen from (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); and (g) efficacy of the agent or its metabolite(s).

Other aspects and features of the disclosure are described more thoroughly below.

### REFERENCE TO COLOR FIGURES

The application file contains at least one figure executed in color. Copies of this patent application publication with color figures will be provided by the Office upon request and payment of the necessary fee.

### BRIEF DESCRIPTION OF THE FIGURES

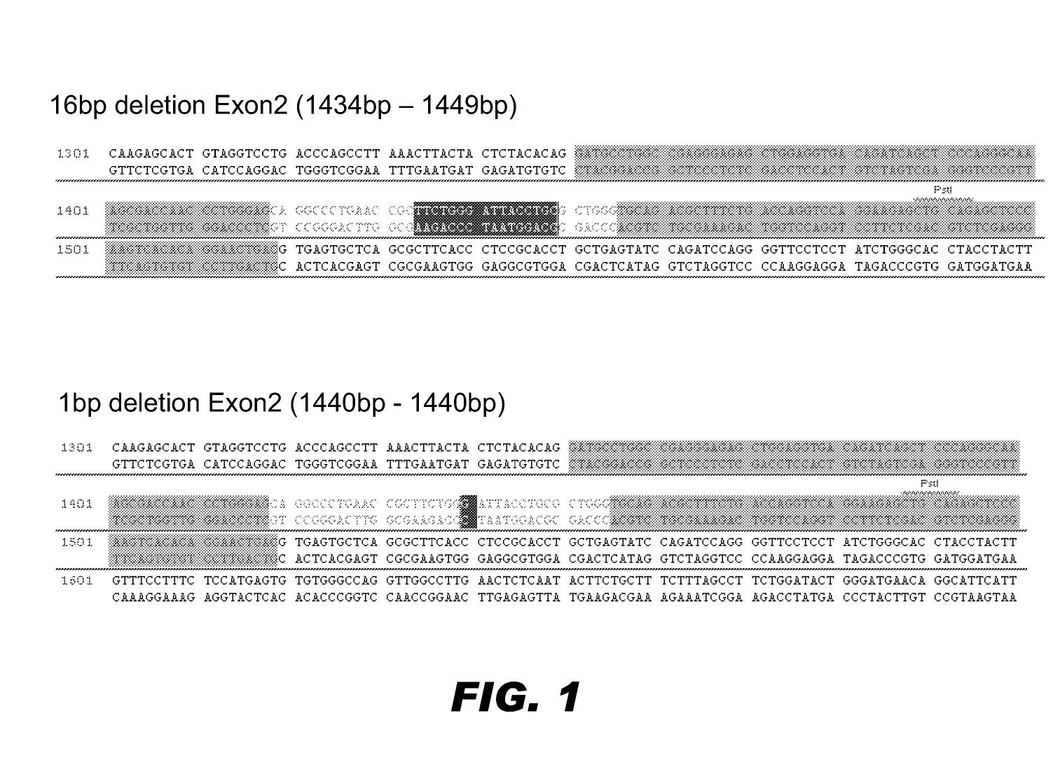

FIG. 1 presents the DNA sequences of two edited ApoE loci. The upper sequence (SEQ ID NO:1) has a 16 by deletion in the target sequence of exon 2, and the lower sequence (SEQ ID NO:2) has a 1 by deletion in the target sequence of exon 2. The exon sequence is shown in green; the target site is presented in yellow, and the deletions are shown in dark blue.

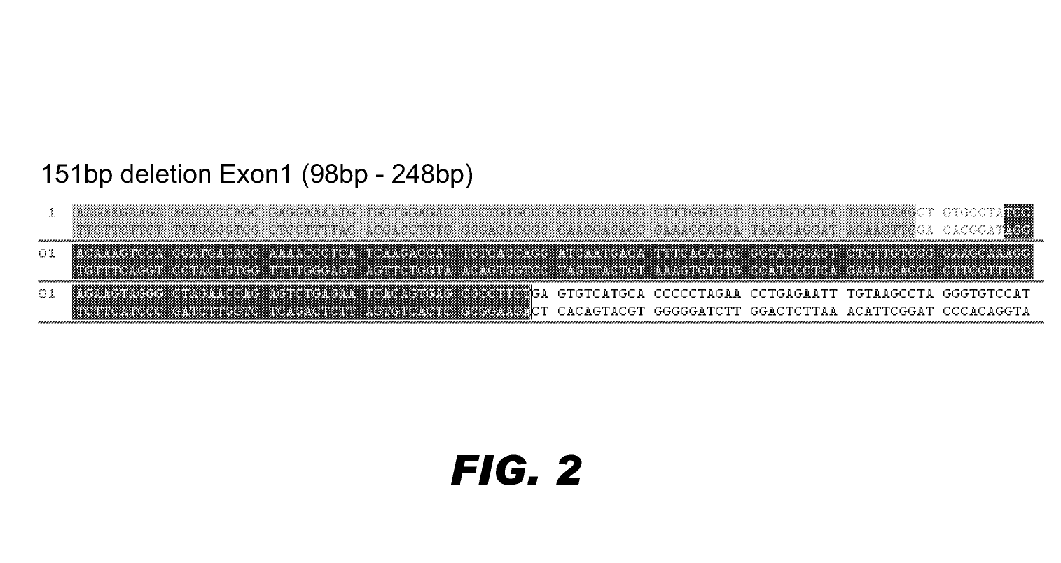

FIG. 2 shows the DNA sequence of an edited leptin locus. Presented is a region of the leptin locus (SEQ ID NO:3) in which 151 by are deleted from exon 1 and intron 1. The exon is shown in green; the target site is presented in yellow, and the deletion is shown in dark blue.

### DETAILED DESCRIPTION OF THE INVENTION

The present disclosure provides a genetically modified animal or animal cell comprising at least one edited chromosomal sequence encoding a protein associated with cardiovascular disease. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein associated with cardiovascular disease generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with cardiovascular disease using targeted zinc finger nuclease technology is rapid, precise, and highly efficient.

### (I) Genetically Modified Animals

One aspect of the present disclosure provides a genetically modified animal in which at least one chromosomal sequence encoding a protein associated with cardiovascular disease has been edited. For example, the edited chromosomal sequence may be inactivated such that the sequence is not transcribed and/or a functional protein associated with cardiovascular disease is not produced. Alternatively, the edited chromosomal sequence may be modified such that it codes for an altered protein associated with cardiovascular disease. For example, the chromosomal sequence may be modified such that at least one nucleotide is changed and the expressed protein associated with cardiovascular disease comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed protein associated with cardiovascular disease comprises a single amino acid deletion or insertion, provided such a protein is functional. The modified protein associated with cardiovascular disease may have altered substrate specificity, altered enzyme activity, altered kinetic rates, and so forth. Furthermore, the edited chromosomal sequence encoding a protein associated with cardiovascular disease may comprise an integrated sequence and/or a sequence encoding an orthologous protein associated with cardiovascular disease may be integrated into the genome of the animal. The genetically modified animal disclosed herein may be heterozygous for the edited chromosomal sequence encoding a protein associated with cardiovascular disease. Alternatively, the genetically modified animal may be homozygous for the edited chromosomal sequence encoding a protein associated with cardiovascular disease.

In one embodiment, the genetically modified animal may comprise at least one inactivated chromosomal sequence encoding a protein associated with cardiovascular disease. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional protein associated with cardiovascular disease is not produced. The inactivated chromosomal sequence comprises no exogenously introduced sequence. Such an animal may be termed a “knockout.” Also included herein are genetically modified animals in which two, three, four, five, six, seven, eight, nine, or ten or more chromosomal sequences encoding proteins associated with cardiovascular disease are inactivated.

In another embodiment, the genetically modified animal may comprise at least one edited chromosomal sequence encoding an orthologous protein associated with cardiovascular disease. The edited chromosomal sequence encoding an orthologous protein associated with cardiovascular disease may be modified such that it codes for an altered protein associated with cardiovascular disease. For example, the edited chromosomal sequence encoding a protein associated with cardiovascular disease may encode a protein associated with cardiovascular disease comprising at least one modification such that an altered version of the protein associated with cardiovascular disease is produced. In some embodiments, the edited chromosomal sequence encoding a protein associated with cardiovascular disease comprises at least one modification such that the altered version of the protein associated with cardiovascular disease produced causes cardiovascular disease. In other embodiments, the edited chromosomal sequence encoding a protein associated with cardiovascular disease comprises at least one modification such that the altered version of the protein associated with cardiovascular disease produced protects against cardiovascular disease. The modification may be a missense mutation in which substitution of one nucleotide for another nucleotide changes the identity of the coded amino acid.

In yet another embodiment, the genetically modified animal may comprise at least one chromosomally integrated sequence. The chromosomally integrated sequence may encode an orthologous protein associated with cardiovascular disease, an endogenous protein associated with cardiovascular disease, or combinations of both. For example, a sequence encoding an orthologous protein or an endogenous protein associated with cardiovascular disease may be integrated into a chromosomal sequence encoding a protein associated with cardiovascular disease such that the chromosomal sequence is inactivated, but wherein the exogenous sequence encoding the orthologous protein or endogenous protein associated with cardiovascular disease may be expressed. In such a case, the sequence encoding the orthologous protein or endogenous protein associated with cardiovascular disease may be operably linked to a promoter control sequence. Alternatively, a sequence encoding an orthologous protein or an endogenous protein associated with cardiovascular disease may be integrated into a chromosomal sequence without affecting expression of a chromosomal sequence. For example, a sequence encoding a protein associated with cardiovascular disease may be integrated into a “safe harbor” locus, such as the Rosa26 locus, HPRT locus, or AAV locus. In one iteration of the disclosure an animal comprising a chromosomally integrated sequence encoding a cardiovascular-related protein may be called a “knock-in”, and it should be understood that in such an iteration of the animal, no selectable marker is present. The present disclosure also encompasses genetically modified animals in which two, three, four, five, six, seven, eight, nine, or ten or more sequences encoding protein(s) associated with cardiovascular disease are integrated into the genome.

The chromosomally integrated sequence encoding a protein associated with cardiovascular disease may encode the wild type form of the protein associated with cardiovascular disease. Alternatively, the chromosomally integrated sequence encoding a protein associated with cardiovascular disease may encode a protein associated with cardiovascular disease comprising at least one modification such that an altered version of the protein associated with cardiovascular disease is produced. In some embodiments, the chromosomally integrated sequence encoding a protein associated with cardiovascular disease comprises at least one modification such that the altered version of the protein associated with cardiovascular disease produced causes cardiovascular disease. In other embodiments, the chromosomally integrated sequence encoding a protein associated with cardiovascular disease comprises at least one modification such that the altered version of the protein associated with cardiovascular disease produced protects against cardiovascular disease.

In an additional embodiment, the genetically modified animal may be a “humanized” animal comprising at least one chromosomally integrated sequence encoding a functional human cardiovascular disease-related protein. The functional human cardiovascular disease-related protein may have no corresponding ortholog in the genetically modified animal. Alternatively, the wild-type animal from which the genetically modified animal is derived may comprise an ortholog corresponding to the functional human cardiovascular disease-related protein. In this case, the orthologous sequence in the “humanized” animal is inactivated such that no functional protein is made and the “humanized” animal comprises at least one chromosomally integrated sequence encoding the human cardiovascular disease-related protein. Those of skill in the art appreciate that “humanized” animals may be generated by crossing a knock out animal with a knock in animal comprising the chromosomally integrated sequence.

In yet another embodiment, the genetically modified animal may comprise at least one edited chromosomal sequence encoding a cardiovascular disease-related protein such that the expression pattern of the protein is altered. For example, regulatory regions controlling the expression of the protein, such as a promoter or transcription binding site, may be altered such that the cardiovascular disease-related protein is over-produced, or the tissue-specific or temporal expression of the protein is altered, or a combination thereof. Alternatively, the expression pattern of the cardiovascular disease-related protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a cardiovascular disease-related protein. The genetically modified animal comprising the lox-flanked chromosomal sequence encoding a cardiovascular disease-related protein may then be crossed with another genetically modified animal expressing Cre recombinase. Progeny animals comprising the lox-flanked chromosomal sequence and the Cre recombinase are then produced, and the lox-flanked chromosomal sequence encoding a cardiovascular disease-related protein is recombined, leading to deletion or inversion of the chromosomal sequence encoding the protein. Expression of Cre recombinase may be temporally and conditionally regulated to effect temporally and conditionally regulated recombination of the chromosomal sequence encoding a cardiovascular disease-related protein.

### (a) Chromosomal Sequences and Proteins Involved in Cardiovascular Disease

Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

By way of example, the chromosomal sequence may comprise, but is not limited to, IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCG5 (ATP-binding cassette, sub-family G (WHITE), member 5), PRDX2 (peroxiredoxin 2), CAPN5 (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINE1 (serpin peptidase inhibitor, clade E (nexin, plasminogen activator inhibitor type 1), member 1), ALB (albumin), ADIPOQ (adiponectin, C1Q and collagen domain containing), APOB (apolipoprotein B (including Ag(x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), VWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp(a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), HMOX1 (heme oxygenase (decycling) 1), APOC3 (apolipoprotein C-III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), IL1A (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor superfamily, member 11b), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C-C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIF1A (hypoxia inducible factor 1, alpha subunit (basic helix-loop-helix transcription factor)), CXCR4 (chemokine (C-X-C motif) receptor 4), PROC (protein C (inactivator of coagulation factors Va and VIIIa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid A1), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomerase G (cyclophilin G)), IL1R1 (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CABIN1 (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member A1), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IL17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, A1 polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member 1B), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide A1), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member A1), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Rac1)), LMNA (lamin NC), CD59 (CD59 molecule, complement regulatory protein), SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin L1), PCNA (proliferating cell nuclear antigen), IGF2 (insulin-like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COL1A2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRF1 (interferon regulatory factor 1), CFLAR (CASP8 and FADD-like apoptosis regulator), CALCA (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PECAM1 (platelet/endothelial cell adhesion molecule), CCL4 (chemokine (C-C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YME1L1 (YME1-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-β-methyltransferase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (B321 placental growth factor), THPO (thrombopoietin), GP6 (glycoprotein VI (platelet)), TACR1 (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADH1C (alcohol dehydrogenase 1C (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHCl (SHC (Src homology 2 domain containing) transforming protein 1), LRP1 (low density lipoprotein receptor-related protein 1), SOCS3 (suppressor of cytokine signaling 3), ADH1B (alcohol dehydrogenase 1B (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor H1), NR112 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQO1 (NAD(P)H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity IIa, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), F11 (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCHE (butyrylcholinesterase), LIPE (lipase, hormone-sensitive), PRDX5 (peroxiredoxin 5), ADORA1 (adenosine A1 receptor), WRN (Werner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CHGA (chromogranin A (parathyroid secretory protein 1)), IAPP (islet amyloid polypeptide), RHO (rhodopsin), ENPP1 (ectonucleotide pyrophosphatase/phosphodiesterase 1), PTHLH (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha-), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISH (cytokine inducible SH2-containing protein), GAST (gastrin), MYOC (myocilin, trabecular meshwork inducible glucocorticoid response), ATP1A2 (ATPase, Na+/K+ transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), HSF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTH (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOH (apolipoprotein H (beta-2-glycoprotein I)), S100A8 (S100 calcium binding protein A8), IL11 (interleukin 11), ALOX15 (arachidonate 15-lipoxygenase), FBLN1 (fibulin 1), NR1H3 (nuclear receptor subfamily 1, group H, member 3), SCD (stearoyl-CoA desaturase (delta-9-desaturase)), GIP (gastric inhibitory polypeptide), CHGB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), HSD11B2 (hydroxysteroid (11-beta) dehydrogenase 2), CALCRL (calcitonin receptor-like), GALNT2 (UDP-N-acetyl-alpha-D-galactosamine:polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adenovirus E1B 19 kDa interacting protein 3), GCKR (glucokinase (hexokinase 4) regulator), S100A12 (S100 calcium binding protein A12), PADI4 (peptidyl arginine deiminase, type IV), HSPA14 (heat shock 70 kDa protein 14), CXCR1 (chemokine (C-X-C motif) receptor 1), H19 (H19, imprinted maternally expressed transcript (non-protein coding)), KRTAP19-3 (keratin associated protein 19-3), IDDM2 (insulin-dependent diabetes mellitus 2), RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNA1C (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase I, alpha), OPRL1 (opiate receptor-like 1), IMPA1 (inositol(myo)-1(or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member B1), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa), EFNB2 (ephrin-B2), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C-C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (F11 receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), KHK (ketohexokinase (fructokinase)), SAT1 (spermidine/spermine N1-acetyltransferase 1), GGH (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RHOB (ras homolog gene family, member B), LY96 (lymphocyte antigen 96), FOXO1 (forkhead box O1), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cell leukemia homeobox 4), NUPR1 (nuclear protein, transcriptional regulator, 1), 15-Sep(15 kDa selenoprotein), CILP2 (cartilage intermediate layer protein 2), TERC (telomerase RNA component), GGT2 (gamma-glutamyltransferase 2), MT-CO1 (mitochondrially encoded cytochrome c oxidase I), and UOX (urate oxidase, pseudogene).

In an additional embodiment, the chromosomal sequence may further be selected from Pon1 (paraoxonase 1), LDLR (LDL receptor), ApoE (Apolipoprotein E), Apo B-100 (Apolipoprotein B-100), ApoA (Apolipoprotein(a)), ApoA1 (Apolipoprotein A1), CBS (Cystathione B-synthase), Glycoprotein IIb/IIb, MTHRF (5,10-methylenetetrahydrofolate reductase (NADPH), and combinations thereof. In one iteration, the chromosomal sequences and proteins encoded by chromosomal sequences involved in cardiovascular disease may be chosen from Cacna1C, Sod1, Pten, Ppar(alpha), Apo E, Leptin, and combinations thereof.

(i) Cacna1C

Cacna1C, calcium channel, voltage dependent L-type, alpha 1C subunit is a subunit of L-type voltage dependent calcium channel encoded by the Cacna1C gene. The Cacna1C gene belongs to a family of genes that provide instructions for making calcium channels. These calcium channels transport positively charged calcium atoms or ions into cells. The channels also play a key role in a cell's ability to generate and transmit electrical signals. Cacna1C produces a calcium channel known as CaV1.2. The CaV1.2 channels are found in many types of cells, but appear to be particularly important for the normal function of heart and brain cells. The CaV1.2 channels open and close at specific times to control the flow of calcium ions into cardiac muscle cells. Calcium channels signal the cardiac muscle to contract and help maintain the heart's normal rhythm by changing the electrical properties of the cardiac muscle cells. Mutations in the Cacna1C gene are responsible for Timothy Syndrome. In Timothy Syndrome, a mutation changes one protein building block used to build the channel, specifically, replacing the amino acid glycine with the amino acid arginine at position 406. Mutations in the Cacna1C gene change the structure of the CaV1.2 channels throughout the body, making the channels stay open longer than usual. This causes a continual flow of calcium ions to flow into the cell at an abnormal rate which can result in the overload of calcium ions within the cardiac muscle cells changing the way the heart beats, causing arrhythmia.

(ii) SOD1

SOD1, superoxide dismutase 1, soluble, provides instructions for making an exzyme called superozide dismutase that is abundant in cells throughout the body. Superoxide dismutase neutralizes supercharged oxygen molecules (superoxide radicals), which can damage cells if their levels are not controlled. These superoxide radicals are byproducts of normal cell processes, particularly energy-producing reactions that occur in the mitochondria. Superoxide dismutase enzyme must bind to copper and zinc in order to function properly. Mutations in the SOD1 gene can lead to amyotrophic lateral sclerosis. Most of the mutations in SOD1 change one of the building blocks or amino acids used to make the enzyme superoxide dismutase. For example in type 1 amyotrophic sclerosis the amino acid alanine is replaced with valine at position 4 within the enzyme. Other types of mutations result in an enzyme of abnormal size. It has been found that brain and spinal cord cells that control muscle movement are particularly sensitive to SOD1 mutations. Additionally, it is thought that some of these mutations result in the altered enzyme causing death of motor neurons.

(iii) Pten

Pten, phosphatase and tensin homolog, provides instructions for making a protein found in almost all bodily tissues. The protein acts as a tumor suppressor, thus, it helps regulate the cell division cycle by keeping cells from growing and dividing too rapidly or in an uncontrolled way. The Pten protein modifies other proteins and lipids by removing phosphate groups, thus, it is called a phosphatase. The Pten enzyme acts as part of a chemical pathway that signals cells to stop dividing and triggers cells to undergo a form of programmed cell death called apoptosis. Pten acts to prevent uncontrolled cell growth that can lead to the formation of tumors as well as helping control migration and adhesion of cells to surrounding tissues. Further, Pten acts to aid in the formation of new blood vessels and plays a role in maintaining the stability of a cell's genetic information. Mutations in the Pten gene have been lined to Cowden syndrome, a rare disorder characterized by multiple noncancerous, tumor-like growths called hamartomas and an increased risk of developing certain cancers; breast cancer; and other disorders. Over 100 mutations have been identified that are believed to be the cause of Cowden syndrome. These mutations are often inherited from a parent and affect all of the body's cells. Pten mutations lead to the production of an enzyme that does not function properly or does not function at all. The dysfunction or nonfunction of the enzyme leads to the body being unable to restrain cell division and signal abnormal cells to die, which can contribute to the development of noncancerous growths called hamartomas and cancerous tumors. Cowden syndrome is linked to a risk of developing breast cancer. Other disorders caused by mutations in the Pten gene are characterized by the development of noncancerous tumor-like growths called hamartomas, including Bannayan-Riley-Rubalcaba syndrome, Proteus syndrome, and Proteus-like syndrome, collectively called PTEN hamartoma tumor syndromes. Mutations in the PTEN gene result in an altered enzyme that has lost its tumor suppressor function, leading to cells that divide uncontrollably, contributing to the growth of cancerous tumors.

(iv) Ppar(alpha)

Ppar(alpha) encodes the peroxisome proliferator-activated receptor alpha. Peroxisome proliferators include hypolipidemic drugs, herbicides, leukotriene antagonists, and plasticizers. Peroxisome proliferators induce an increase in the size and number of peroxisomes, which are subcellular organelles found in plants and animals that contain enzymes for respiration and for cholesterol and lipid metabolism. PPARs are specific receptors believed to mediate peroxisome proliferators.

(v) Apo E

Apo E provides instructions for making a protein called apolipoprotein E. This protein combines with lipids in the body to form lipoproteins. Lipoproteins are responsible for packaging cholesterol and other fats and carrying them through the bloodstream. A major component of a specific type of lipoprotein called very low-density lipoproteins (VLDLs) is apolipoprotein E. Extra cholesterol from the blood is carried over the liver for processing by VLDLs. The maintenance of normal cholesterol levels is essential for the prevention of cardiovascular diseases. Apo E has three different alleles called e2, e3, and e4, with e3 being the most common. Those with the e4 version of ApoE have an increased risk for late-onset Alzheimer disease. A higher risk is associated with the inheritance of two copies of the e4 allele of the gene. Variants of ApoE have been studies as risk factors for many different conditions. Specifically, ApoE alleles have been shown to influence the risk of developing cardiovascular disease and atherosclerosis. The e2 allele has been shown to increase the risk of a rare condition called hyperlipoporoteinemia type II, with many of the people afflicted with this condition having two e2 alleles. ApoE has also been identified as a potential risk factor for age-related macular degeneration.

(vi) LEP

LEP or Leptin, encodes a protein that is secreted by white adipocytes and which plays a major role in the regulation of body weight. The leptin protein acts through the leptin receptor and functions as part of a signaling pathway that can inhibit food intake and/or regulate energy expenditure to maintain constancy of the adipose mass. Leptin also have several endocrine functions and is involved in the regulation of immune and inflammatory responses, hematopoiesis, angiogenesis, and wound healing. Mutations in LEP and/or its regulatory regions can cause severe obesity, morbid obesity with hypergonadism, and type 2 diabetes.

The identity of a protein involved in cardiovascular disease whose chromosomal sequence is edited can and will vary. For example, the edited chromosomal sequence may encode any of the foregoing chromosomal sequences or proteins encoded by the sequences that are associated with cardiovascular disease or any combination of such sequences. In this regard, the genetically modified animal or cell may comprise one, two, three, four, five, six, seven, eight, nine, or ten or more edited chromosomal sequences encoding a protein associated with cardiovascular disease. Table A details non-limiting examples of chromosomal sequences that may be edited in accordance with the present disclosure. For example, those rows having no entry in the “Protein Sequence” column indicate a genetically modified animal in which the sequence specified in that row under “Inactivated Sequence” is inactivated (i.e., a knock-out). Subsequent rows indicate single or multiple knock-outs with knock-ins of one or more integrated orthologous sequences, as indicated in the “Protein Sequence” column.”

TABLE A

| Inactivated Sequence | Protein Sequence |
|---|---|
| canca1c | none |
| sod1 | none |
| pten | none |
| ppar(alpha) | none |
| apoe | none |
| lep | none |
| canca1c | CANCA1C |
| sod1 | SOD1 |
| pten | PTEN |
| ppar(alpha) | PPAR(alpha) |
| apoe | APOE |
| lep | LEP |
| canca1c, sod1 | CANCA1C, SOD1 |
| canca1c, pten | CANCA1C, PTEN |
| canca1c, ppar(alpha) | CANCA1C, PPAR(alpha) |
| cacna1c, apoe | CANCA1C, APOE |
| cacna1c, lep | CACNA1C, LEP |
| sod1, pten | SOD1, PTEN |
| sod1, ppar(alpha) | SOD1, PPAR (alpha) |
| sod1, apoe | SOD1, APOE |
| sod1, lep | SOD1, LEP |
| pten, apoe | PTEN, APOE |
| pten, lep | PTEN, LEP |
| pten, ppar(alpha) | PTEN, PPAR(alpha) |
| ppar(alpha), apoe | PPAR(alpha), APOE |
| ppar(alpha), lep | PPAR(alpha), LEP |
| apoe, lep | APOE, LEP |
| canca1c, sod1, pten | CANCA1C, SOD1, PTEN |
| canca1c, sod1, ppar(alpha) | CANCA1C, SOD1, PPAR(alpha) |
| cacna1c, sod1, apoe | CANCA1C, SOD1, APOE |
| cacna1c, sod1, lep | CACNA1C, SOD1, LEP |
| canca1c, pten, ppar(alpha) | CANCA1C, PTEN, PPAR(alpha) |
| cacna1c, pten, apoe | CACNA1C, PTEN, APOE |
| cacna1c, pten, lep | CACNA1C, PTEN, LEP |
| sod1, pten, ppar(alpha) | SOD1, PTEN, PPAR(alpha) |
| ppar(alpha), apoE, lep | PPAR(alpha), APOE, LEP |
| pten, apoe, lep | PTEN, APOE, LEP |
| sod1, apoe, lep | SOD1, APOE, LEP |
| canca1c, apoe, lep | CACNA1C, APOE, LEP |
| canca1c, sod1, pten, ppar(alpha) | CANCA1C, SOD1, PTEN, PPAR(alpha) |
| cacna1c, sod1, pten, apoe | CACNA1C, SOD1, PTEN, APOE |
| cacna1c, sod1, pten, lep | CACNA1C, SOD1, PTEN, LEP |
| cacna1c, sod1, ppar(alpha), apoe | CACNA1C, SOD1, PPAR(alpha), APOE |
| cacna1c, sod1, ppar(alpha), lep | CACNA1C, SOD1, PPAR(alpha), LEP |
| cacna1c, ppar(alpha), pten, lep, | CACNA1C, PPAR(alpha), PTEN, LEP |
| cacna1c, ppar(alpha), pten, apoe | CACNA1C, PPAR(alpha), PTEN, APOE |
| cacna1c, apoe, lep, pten | CACNA1C, APOE, LEP, PTEN |
| cacna1c, apoe, lep, ppar(alpha) | CACNA1C, APOE, LEP, PPAR(alpha) |
| cacna1c, apoe, lep, sod1 | CACNA1C, APOE, LEP, SOD1 |
| apoe, lep, sod1, pten | APOE, LEP, SOD1, PTEN |
| apoe, lep, sod1, ppar(alpha) | APOE, LEP, SOD1, PPAR(alpha) |
| sod1, ppar(alpha), apoe, pten | SOD1, PPAR(alpha), APOE, PTEN |
| sod1, ppar(alpha), lep, pten | SOD1, PPAR(alpha), LEP, PTEN |
| pten, apoe, ppar(alpha), lep | PTEN, APOE, PPAR(alpha), LEP |
| cacna1c, sod1, pten, ppar(alpha), apoe | CACNA1C, SOD1, PTEN, PPAR(alpha), APOE |
| cacna1, sod1, pten, ppar(alpha), lep | CACNA1C, SOD1, PTEN, PPAR(alpha), LEP |
| sod1, pten, ppar(alpha), lep, apoe | SOD1, PTEN, PPAR(alpha), LEP, APOE |
| cacna1c, pten, ppar(alpha), lep, apoe | CACNA1C, PTEN, PPAR(alpha), LEP, APOE |
| cacna1c, sod1, pten, apoe, lep | CACNA1C, SOD1, PTEN, APOE, LEP |
| cacna1c, sod1, ppar(alpha), apoe, lep | CACNA1C, SOD1, PPAR(alpha), APOE, LEP |
| cacna1c, sod1, pten, ppar(alpha), apoe, lep | CACNA1C, SOD1, PTEN, PPAR(alpha), APOE, LEP |

### (b) Animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of suitable rat strains include Dahl Salt-Sensitive, Fischer 344, Lewis, Long Evans Hooded, Sprague-Dawley, and Wistar. In another iteration of the invention, the animal does not comprise a genetically modified mouse. In each of the foregoing iterations of suitable animals for the invention, the animal does not include exogenously introduced, randomly integrated transposon sequences.

(c) Proteins Associated with Cardiovascular Disease

The protein involved in cardiovascular disease may be from any of the animals listed above. Furthermore, the protein involved in cardiovascular disease may be a human protein. Additionally, the protein involved in cardiovascular disease may be a bacterial, fungal, or plant protein. The type of animal and the source of the protein can and will vary. The protein may be endogenous or exogenous (such as an orthologous protein). As an example, the genetically modified animal may be a rat, cat, dog, or pig, and the orthologous protein involved in cardiovascular disease may be human. Alternatively, the genetically modified animal may be a rat, cat, or pig, and the orthologous protein involved in cardiovascular disease may be canine. One of skill in the art will readily appreciate that numerous combinations are possible.

Additionally, the cardiovascular disease-related gene may be modified to include a tag or reporter gene or genes as are well-known. Reporter genes include those encoding selectable markers such as chloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalamal), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

### (II) Genetically Modified Cells

A further aspect of the present disclosure provides genetically modified cells or cell lines comprising at least one edited chromosomal sequence involved in cardiovascular disease. The genetically modified cell or cell line may be derived from any of the genetically modified animals disclosed herein. Alternatively, the chromosomal sequence involved in cardiovascular disease may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double stranded breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TR1 cells. For an extensive list of mammalian cell lines, those of ordinary skill in the art may refer to the American Type Culture Collection catalog (ATCC®, Mamassas, Va.).

In another embodiment, the chromosomal sequence involved in cardiovascular disease may be targeted for disruption in a rat strain chosen from Dahl Salt-Sensitive, Fisher 344, Lewis, Long Evans Hooded, Sprague-Dawley, and Wistar.

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

### (III) Zinc Finger-Mediated Genome Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

### (a) Zinc Finger Nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

### (i) Zinc Finger Binding Domain

Zinc finger binding domains may be engineered to recognize and bind to any nucleic acid sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; and Choo et al. (2000) Curr. Opin. Struct. Biol. 10:411-416. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (see, for example, Biochemistry 2002, 41, 7074-7081).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

An exemplary zinc finger DNA binding domain recognizes and binds a sequence having at least about 80% sequence identity with a sequence chosen from SEQ ID NOs: 4, 5, 6, and 7. In other embodiments, the sequence identity may be about 81%, 82%, 83%, 84%, 85%, 86%, 87%, 88%, 89%, 90%, 91%, 92%, 93%, 94%, 95%, 96%, 97%, 98%, 99%, or 100%.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from Ito K in one cleavage monomer to produce an engineered cleavage monomer designated “E490K:I538K” and by mutating positions 486 from Q to E and 499 from Ito L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Donor Polynucleotide

The method for editing chromosomal sequences involved in cardiovascular disease may further comprise introducing at least one donor polynucleotide comprising a sequence encoding a protein involved in cardiovascular disease into an embryo or cell. A donor polynucleotide comprises at least three components: the sequence encoding the protein involved in cardiovascular disease, an upstream sequence and a downstream sequence. The sequence encoding the protein is flanked by the upstream and downstream sequence, wherein the upstream and downstream sequences share sequence similarity with either side of the site of integration in the chromosome. In various embodiments, the chromosomal sequence involved in cardiovascular disease is chosen from Cacna1C, Sod1, Pten, Ppar(alpha), ApoE, Leptin, and combinations thereof.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding an orthologous protein involved in cardiovascular disease may be a BAC.

The sequence of the donor polynucleotide that encodes the protein involved in cardiovascular disease may include a coding (i.e., exon) sequence, as well as intron sequences and upstream regulatory sequences (such as, e.g., a promoter). Depending upon the identity and the source of the protein involved in cardiovascular disease, the size of the sequence encoding a protein involved in cardiovascular disease will vary. For example, the sequence encoding a protein involved in cardiovascular disease may range in size from about 1 kb to about 5,000 kb.

The donor polynucleotide also comprises upstream and downstream sequences flanking the chromosomal sequence involved in cardiovascular disease. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 by to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 by to about 2000 bp, about 600 by to about 1000 bp, or more particularly about 700 by to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a chromosomal sequence involved in cardiovascular disease, a double stranded break introduced into the chromosomal sequence by the zinc finger nuclease is repaired, via homologous recombination with the donor polynucleotide, such that the sequence encoding a protein involved in cardiovascular disease is integrated into the chromosome. The presence of a double-stranded break facilitates integration of the sequence into the chromosome. A donor polynucleotide may be physically integrated or, alternatively, the donor polynucleotide may be used as a template for repair of the break, resulting in the introduction of the sequence encoding a protein involved in cardiovascular disease as well as all or part of the upstream and downstream sequences of the donor polynucleotide into the chromosome. Thus, endogenous chromosomal sequence may be converted to the sequence of the donor polynucleotide.

### (c) Optional Exchange Polynucleotide

The method for editing chromosomal sequences involved in cardiovascular disease may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85, 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 by to about 10,000 by in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 by in length. In other embodiments, the sequence in the exchange polynucleotide may be about 5500, 6000, 6500, 6000, 6500, 7000, 7500, 8000, 8500, 9000, 9500, or 10,000 by in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (d) Delivery of Nucleic Acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo of the species of interest.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides may be introduced at the same time. Alternatively, each nucleic acid encoding a zinc finger nuclease, as well as the optional donor (or exchange) polynucleotides, may be introduced sequentially.

### (e) Culturing the Embryo or Cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence involved in cardiovascular disease in every cell of the body. Preferably, the chromosomal sequence involved in cardiovascular disease is chosen from Cacna1C, Sod1, Pten, Ppar(alpha), ApoE, Leptin, and combinations thereof.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by the error-prone non-homologous end-joining DNA repair process. Consequently, a deletion, insertion or nonsense mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor (or exchange) polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified). Preferably, the chromosomal sequence involved in cardiovascular disease is chosen from Cacna1C, Sod1, Pten, Ppar(alpha), ApoE, Leptin, and combinations thereof.

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated Cacna1C chromosomal sequence may be crossed with animal B comprising a chromosomally integrated sequence encoding the human Cacna1C protein to give rise to a “humanized” Cacna1C offspring comprising both the inactivated Cacna1C chromosomal sequence and chromosomally integrated human Cacna1C sequence. Similarly, an animal comprising an inactivated Sod1 chromosomal sequence may be crossed with an animal comprising a chromosomally integrated sequence encoding the human Sod1 protein to generate “humanized” Sod1 offspring. Moreover, a humanized Cacna1C animal may be crossed with a humanized Sod1 animal to create a humanized Cacna1C/Sod1 animal. Those of skill in the art will appreciate that many combinations are possible.

In other embodiments, an animal comprising an edited chromosomal sequence disclosed herein may be crossbred to combine the edited chromosomal sequence with other genetic backgrounds. By way of non-limiting example, other genetic backgrounds may include wild-type genetic backgrounds, genetic backgrounds with deletion mutations, genetic backgrounds with another targeted integration and genetic backgrounds with non-targeted integrations. Suitable integrations may include without limit nucleic acids encoding drug transporter proteins, Mdr protein, and the like.

### (IV) Applications

A further aspect of the present disclosure encompasses a method for assessing the effect(s) of an agent. Suitable agents include without limit pharmaceutically active ingredients, drugs, food additives, pesticides, herbicides, toxins, industrial chemicals, household chemicals, and other environmental chemicals. For example, the effect(s) of an agent may be measured in a “humanized” genetically modified animal, such that the information gained therefrom may be used to predict the effect of the agent in a human. In general, the method comprises contacting a genetically modified animal comprising at least one inactivated chromosomal sequence encoding a protein from a chromosomal sequence involved in cardiovascular disease and at least one chromosomally integrated sequence encoding a orthogolous protein involved in cardiovascular disease with the agent, and comparing results of a selected parameter to results obtained from contacting a wild-type animal with the same agent. Selected parameters include but are not limited to (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); (g) efficacy of the agent or its metabolite(s); (h) disposition of the agent or its metabolite(s); and (i) extrahepatic contribution to metabolic rate and clearance of the agent or its metabolite(s).

Also provided are methods to assess the effect(s) of an agent in an isolated cell comprising at least one edited chromosomal sequence involved in cardiovascular disease, as well as method of using lysates of such cells (or cells derived from a genetically modified animal disclosed herein) to assess the effect(s) of an agent. For example, the role of a particular protein involved in cardiovascular disease in the metabolism of a particular agent may be determined using such methods. Similarly, substrate specificity and pharmacokinetic parameter may be readily determined using such methods. Those of skill in the art are familiar with suitable tests and/or procedures.

Yet another aspect encompasses a method for assessing the therapeutic efficacy of a potential gene therapy. That is, a chromosomal sequence encoding a cardiovascular disease-related protein may be modified to ameliorate symptoms of a cardiovascular disease. In particular, the method comprises editing a chromosomal sequence encoding a cardiovascular disease-related protein such that an altered protein product is produced. The genetically modified animal may further be exposed to a potential therapeutic agent and behavioral, cellular, and/or molecular responses measured and compared to those of a wild-type animal exposed to the same agent. Consequently, information relating to the therapeutic potential of the cardiovascular disease-related gene therapy regime may be assessed.

Still yet another aspect encompasses a method of generating a cell line or cell lysate using a genetically modified animal comprising an edited chromosomal sequence encoding a cardiovascular disease-related protein. An additional other aspect encompasses a method of producing purified biological components using a genetically modified cell or animal comprising an edited chromosomal sequence encoding a cardiovascular disease-related protein. Non-limiting examples of biological components include antibodies, cytokines, signal proteins, enzymes, receptor agonists and receptor antagonists. For example, human apolipoprotein E (ApoE) is a ligand for the low-density-lipoprotein receptors (LDLRs), which activate cell signaling in the central nervous system (Rogers & Weeber (2008) Neuron Glia Biol. 4(3): 259-70). ApoE mutation or overexpression is associated with increased plasma cholesterol levels, coronary heart disease (atherosclerosis, the leading cause of death globally), and Alzheimer's disease. The apoE4 allele appears to be a contributing factor to 40-65% of sporadic and familial cases of Alzheimer's disease (Corder et al. (1993) Science 261:921-923). The allele apoE4 is also predicts poor outcomes in cases of acute head trauma or stroke. (Slooter et al. (1997) JAMA 277:818-821; and Nicoll et al. (1996) Neuropathol. Appl. Neurobiol. 22:515-517). Mouse mutants with disrupted ApoE genes display a variety of phenotypes including i) increase in total plasma cholesterol levels; ii) fatty deposits in the aorta; iii) increased triglycerides; iv) brain lesions containing cholesterol, lipid and foam cells; v) synaptic damage; vi) damaged capacity for long-term potentiation in neurons; and vii) behavioral abnormalities including altered stress responses, impaired learning and memory (Gozes, (2004) J. Molec. Neurosci. 23(3) 149-150. In addition, apoE knockout mice develop spontaneous atherosclerosis have hyperglycemia, aortic stiffening, and cardiac hypertrophy. Several drugs and metabolites affect the apoE phenotype, including angiotensin II, nitric oxide, estrogen, and simvastatin (Wang, (2005) Neurobiol. Aging, 26(3): 309-16.). Atheroslcerosis appears to be a chronic inflammatory disease initiated by hypercholeterolemia, and further, ApoE mediates estradiol effects on nerve repair, which promotes neuronal plasticity (Struble et. al (2008) Front. Biosci, 13:5387-405). Thus, potential therapeutic agents can be screened for effectiveness in ameliorating phenotypes associated with ApoE mutations (mutations including gene knockouts, partial knockouts, overexpression, or disease allele expression, and in combination with other gene knockouts or transgene expression) that are related to a cardiovascular disease or condition. Disease phenotypes may include behavioral, electrophysiological, neurochemical, biochemical, or cellular dysfunctions which can be evaluated using any of a number of well-known diagnostic tests or assays. Similarly, genetically modified animals, embryos or cells containing a ZFN-mediated genetic modification of one or more other cardiovascular-related proteins as set forth herein, including for example Cacna1C, Sod1, Pten, Ppar(alpha), and Leptin, can be used in methods and bioassays to evaluate potential therapeutic agents for effectiveness in ameliorating phenotypes associated with the specific mutations (including gene knockouts, partial knockouts, overexpression, or disease allele expression, and in combination with other gene knockouts or transgene expression) that are related to a cardiovascular disease or condition.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “chromosomal sequence involved in cardiovascular disease” refers to a chromosomal sequence which has been identified to be a cause or factor in the development of cardiovascular disease and related complications. Cardiovascular disease specifically targets the heart and blood vessels. Exemplary chromosomal sequences involved in cardiovascular disease include, but are not limited to, Cacna1C, Sod1, Pten, Ppar(alpha), ApoE, and Leptin. Any chromosomal sequence known to be involved in cardiovascular disease is included within the scope of the present invention.

The term “a protein encoded by a chromosomal sequence involved in cardiovascular disease” or “a protein involved in cardiovascular disease” refers to a protein that has been encoded by a chromosomal sequence identified to be a cause or factor in the development of cardiovascular disease and related complications. Exemplary proteins involved in cardiovascular disease include, but are not limited to, Calcium channel, voltage dependent, L type, alpha 1C subunit, encoded by the Cacna1C chromosomal sequence; superoxide dismutase [Cu—Zn] (Sod1), an enzyme encoded by Sod1 chromosomal sequence; phosphatase and tensin homolog (Pten), encoded by Pten chromosomal sequence; and peroxisome proliferator-activated receptor alpha, a nuclear receptor protein encoded by Ppar(alpha). Any type of protein involved in cardiovascular disease is included in the scope of the present invention including, but not limited to, structural proteins, enzyme and catalytic proteins, transport proteins, hormonal proteins, contractile proteins, storage proteins, genetic proteins, defense proteins, and receptor proteins.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ-FCARDIOVASCULAR DISEASEB+GenBank CDS translations-FSwiss protein+Spucardiovascular diseaseate+PIR. Details of these programs can be found on the GenBank website. With respect to sequences described herein, the range of desired degrees of sequence identity is approximately 80% to 100% and any integer value there between. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention. I

### Example 1

### Identification of ZFNs that Edit the ApoE Locus

The ApoE gene was chosen for zinc finger nuclease (ZFN) mediated genome editing. ZFNs were designed, assembled, and validated using strategies and procedures previously described (see Geurts et al. Science (2009) 325:433). ZFN design made use of an archive of pre-validated 1-finger and 2-finger modules. The rat ApoE gene region (NM_{—}138828) was scanned for putative zinc finger binding sites to which existing modules could be fused to generate a pair of 4-, 5-, or 6-finger proteins that would bind a 12-18 by sequence on one strand and a 12-18 by sequence on the other strand, with about 5-6 by between the two binding sites.

Capped, polyadenylated mRNA encoding each pair of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the CeI-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease CeI-1, and the cleavage products can be resolved by gel electrophoresis. This assay revealed that the ZFN pair targeted to bind 5′ aaGCGGTTCAGGGCCTGctcccagggtt-3′ (SEQ ID NO: 4; contact sites in uppercase) and 5′ ggGATTACCTGcGCTGGGtgcagacgct-3′ (SEQ ID NO: 5) cleaved within the ApoE locus

### Example 2

### Editing the ApoE Locus in Rat Embryos

Capped, polyadenylated mRNA encoding the active pair of ZFNs was microinjected into fertilized rat embryos using standard procedures (e.g., see Geurts et al. (2009) supra). The injected embryos were either incubated in vitro, or transferred to pseudopregnant female rats to be carried to parturition. The resulting embryos/fetus, or the toe/tail of clip live born animals were harvested for DNA extraction and analysis. DNA was isolated using standard procedures. The targeted region of the ApoE locus was PCR amplified using appropriate primers. The amplified DNA was subcloned into a suitable vector and sequenced using standard methods. FIG. 1 presents two edited ApoE loci. One animal had a 16 by deletion in the target sequence of exon 2, and a second animal had a 1 by deletion in the target sequence of exon 2. These deletions disrupt the reading frame of the ApoE coding region.

### Example 3

### Identification of ZFNs that Edit the Leptin Locus

ZFNs that target and cleave the leptin gene in rat were identified essentially as described above. The rat leptin gene (NM_{—}013076) was scanned for putative zinc finger binding sites. ZFNs were assembled and tested essentially as described in Example 1. This assay revealed that the ZFN pair targeted to bind 5′-gtGGATAGGCACAGcttgaacataggac-3′ (SEQ ID NO: 6; contact sites in uppercase) and 5′ aaGTCCAGGATGACACCaaaaccctcat-3′ (SEQ ID NO: 7) cleaved within the leptin locus

### Example 4

### Editing the Leptin Locus in Rat Embryos

Rat embryos were microinjected with mRNA encoding the active pair of leptin ZFNs essentially as described in Example 2. The injected embryos were incubated and DNA was extracted from the resultant animals. The targeted region of the leptin locus was PCR amplified using appropriate primers. The amplified DNA was subcloned into a suitable vector and sequenced using standard methods. FIG. 2 presents an edited leptin locus, in which a 151 by region was deleted from the 3′ end of exon 1 and the 5′ end of intron 1.

### Example 5

### Editing the Pten Locus in Rat Embryos

ZFNs that target and cleave the Pten locus in rats were designed and tested for activity essentially as described above in Example 1. An active pair of ZFNs was identified. The DNA binding sites were 5′-CCCCAGTTTGTGGTCtgcca-3′ SEQ ID NO:8) and 5′-gcTAAAGGTGAAGATCTA-3′ (SEQ ID NO:9). Capped, polyadenylated mRNA encoding the active pair may be microinjected into rat embryos and the resultant embryos may b analyzed as described in Example 2. Accordingly, the Pten locus may be edited to contain a deletion or an insertion such that the coding region is disrupted and no functional gene product is made.

### Example 6

### Genome Editing of Canca1C in Model Organism Cells

Zinc finger nuclease (ZFN)-mediated genome editing may be tested in the cells of a model organism such as a rat using ZFN that binds to the chromosomal sequences of a cardiovascular-related gene of the rat cell such as Canca1C, Sod1, Pten, Ppar(alpha), and combinations thereof. The particular chromosomal sequence involved in cardiovascular disease to be edited may be a gene having identical DNA binding sites to the DNA binding sites of the corresponding human homologue of the gene. Capped, polyadenylated mRNA encoding the ZFN may be produced using known molecular biology techniques, including, but not limited to, a technique substantially similar to the technique described in Science (2009) 325:433, which is incorporated by reference herein in its entirety. The mRNA may be transfected into rat cells as well as human K562 cells, assuming the K562 cells have identical DNA binding sites. Control cells may be injected with mRNA encoding GFP.

The frequency of ZFN-induced double strand chromosomal breaks may be determined using the CeI-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease CeI-1, and the cleavage products can be resolved by gel electrophoresis. The relative intensity of the cleavage products compared with the parental band is a measure of the level of CeI-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected cognition-related gene locus in human and rat cells using a ZFN.

### Example 7

### Genome Editing of Canca1C in Model Organism Embryos

The embryos of a model organism such as a rat may be harvested using standard procedures and injected with capped, polyadenylated mRNA encoding a ZFN similar to that described in Example 6. The rat embryos may be at the single cell stage when microinjected. Control embryos may be injected with 0.1 mM EDTA. The frequency of ZFN-induced double strand chromosomal breaks may be estimated using the CeI-1 assay as described in Example 6. The cutting efficiency may be estimated using the CEI-1 assay results.

The development of the embryos following microinjection may be assessed. Embryos injected with a small volume ZFN mRNA may be compared to embryos injected with EDTA to determine the effect of the ZFN mRNA on embryo survival to the blastula stage.

The table below presents the amino acid sequences of helices of the active ZFNs.

| | | SEQ |
|---|---|---|
| Name | Sequence of Zinc Finger Helices | ID NO: |
| ApoE | RSDALSV DSSHRTR RSDNLSE TSGSLTR | 10 |
| | RSDDLTR | |
| ApoE | RSDHLSR QSSDLRR RSDVLSA DRSNRIK | 11 |
| | TSSNLSR | |
| Leptin | RSDALSE QNATRTK RSDYLST QNAHRKT | 12 |
| Leptin | DQSTLRN DRSNLSR TSANLSR RSDNLSE | 13 |
| | DRSALAR | |

What is claimed is:

1. A genetically modified animal comprising at least one edited chromosomal sequence encoding a protein involved in cardiovascular disease.

2. The genetically modified animal of claim 1, wherein the edited chromosomal sequence is inactivated, modified, or comprises an integrated sequence.

3. The genetically modified animal of claim 1, wherein the edited chromosomal sequence is inactivated such that no functional cardiovascular-related protein is produced.

4. The genetically modified animal of claim 3, wherein the inactivated chromosomal sequence comprises no exogenously introduced sequence.

5. The genetically modified animal of claim 3, further comprising at least one chromosomally integrated sequence encoding a functional protein involved in cardiovascular disease.

6. The genetically modified animal of 4, wherein the chromosomal sequence encoding a protein involved in cardiovascular disease is chosen from Cacna1C, Sod1, Pten, Ppar(alpha), ApoE, Leptin, and combinations thereof.

7. The genetically modified animal of claim 1, further comprising a conditional knock-out system for conditional expression of the cardiovascular disease-related protein.

8. The genetically modified animal of claim 1, wherein the edited chromosomal sequence comprises an integrated reporter sequence.

9. The genetically modified animal of claim 1, wherein the animal is heterozygous or homozygous for at least one edited chromosomal sequence.

10. The genetically modified animal of claim 1, wherein the animal is an embryo, a juvenile, or an adult.

11. The genetically modified animal of claim 1, wherein the animal is chosen from a bovine, canine, equine, feline, ovine, porcine, non-human primate, and rodent.

12. The genetically modified animal of claim 1, wherein the animal is rat.

13. The genetically modified animal of claim 1, wherein the animal is rat and the othologous protein involved in cardiovascular disease is human.

14. A non-human embryo, the embryo comprising at least one RNA molecule encoding a zinc finger nuclease that recognizes a chromosomal sequence involved in cardiovascular disease, and, optionally, at least one donor polynucleotide comprising a sequence encoding a protein encoded by the chromosomal sequence involved in cardiovascular disease.

15. The non-human embryo of 14, wherein the protein encoded by the chromosomal sequence involved in cardiovascular disease is chosen from Cacna1C, Sod1, Pten, Ppar (alpha), ApoE, Leptin, and combinations thereof.

16. The non-human embryo of claim 14, wherein the embryo is chosen from bovine, canine, equine, feline, ovine, porcine, primate, or rodent.

17. The non-human embryo of claim 14, wherein the embryo is a rat and the protein is an orthologous protein encoded by a chromosomal sequence involved in cardiovascular disease is human.

18. A genetically modified cell, the cell comprising at least one edited chromosomal sequence involved in cardiovascular disease.

19. The genetically modified cell of claim 18, wherein the edited chromosomal sequence is inactivated, modified, or comprises an integrated sequence.

20. The genetically modified cell of claim 18, wherein the edited chromosomal sequence is inactivated such that no functional cardiovascular-related protein is produced.

21. The genetically modified cell of claim 20, wherein the inactivated chromosomal sequence comprises no exogenously introduced sequence.

22. The genetically modified cell of claim 21, further comprising at least one chromosomally integrated sequence encoding a functional protein involved in cardiovascular disease.

23. The genetically modified cell 18, wherein the protein encoded by the chromosomal sequence involved in cardiovascular disease is chosen from Cacna1C, Sod1, Pten, Ppar(alpha) Apo E, Leptin, and combinations thereof.

24. The genetically modified cell of claim 18, further comprising a conditional knock-out system for conditional expression of the cardiovascular disease-related protein.

25. The genetically modified cell of claim 18, wherein the edited chromosomal sequence comprises an integrated reporter sequence.

26. The genetically modified cell of claim 18, wherein the cell is heterozygous or homozygous for the at least one edited chromosomal sequence.

27. The genetically modified cell of claim 18, wherein the cell is chosen from bovine, canine, equine, feline, human, ovine, porcine, non-human primate, and rodent origin.

28. The genetically modified cell of claim 18, wherein the cell is of rat origin and the orthologous protein encoded by a chromosomal sequence involved in cardiovascular disease is human.

29. A zinc finger nuclease, the zinc finger nuclease comprising:
a) a zinc finger DNA binding domain that binds a sequence having at least about 80% sequence identity with a sequence chosen from SEQ ID NOs:4, 5, 6 and 7.
b) a cleavage domain.

30. The zinc finger nuclease of claim 29, wherein the sequence identity is at least about 85%, 90%, 95%, or 100%.

31. The zinc finger nuclease of claim 29, wherein the DNA binding domain comprises at least 3 zinc finger recognition regions.

32. The zinc finger nuclease of claim 29, wherein the cleavage domain is a wild-type or an engineered FokI cleavage domain.

33. A nucleic acid encoding the zinc finger nuclease of claim 29.

34. A nucleic acid sequence bound by a zinc finger nuclease, the nucleic acid sequence having at least about 80% sequence identity with a sequence chosen from SEQ ID NOs: 4, 5, 6 and 7.

35. A method for assessing the effect of an agent in an animal, the method comprising contacting a genetically modified animal, comprising at least one edited chromosomal sequence encoding a protein involved in cardiovascular disease, with the agent and comparing results of a selected parameter to results obtained from contacting a wild-type animal with the same agent, wherein the selected parameter is chosen from:
a) rate of elimination of the agent or its metabolite(s);
b) circulatory levels of the agent or its metabolite(s);
c) bioavailability of the agent or its metabolite(s);
d) rate of metabolism of the agent or its metabolite(s);
e) rate of clearance of the agent or its metabolite(s);
f) toxicity of the agent or its metabolite(s); and
g) efficacy of the agent or its metabolite(s).

36. The method of claim 35, wherein the agent is a pharmaceutically active ingredient, a drug, a toxin, or a chemical.

37. The method of claim 35, wherein the at least one edited chromosomal sequence is inactivated such that the protein involved in cardiovascular disease is not produced or is not functional, and wherein the animal further comprises at least one chromosomally integrated sequence encoding an ortholog of the protein involved in cardiovascular disease.

38. The method of claim 35, wherein the protein involved in cardiovascular disease is chosen from Cacna1C, Sod1, Pten, Ppar(alpha), Apo E, Leptin, and combinations thereof.


SEQUENCE LISTING (13 sequences)

SEQ ID NO: 1 - DNA, 300 bp, Rattus rattus
caagagcactgtaggtcctgacccagccttaaacttactactctacacaggatgcctggccgagggagagctggaggtgacagatcagctcccagggcaaagcgaccaaccctgggagcaggccctgaaccgcttctgggattacctgcgctgggtgcagacgctttctgaccaggtccaggaagagctgcagagctcccaagtcacacaggaactgacgtgagtgctcagcgcttcaccctccgcacctgctgagtatccagatccaggggttcctcctatctgggcacctacctactt

SEQ ID NO: 2 - DNA, 400 bp, Rattus rattus
caagagcactgtaggtcctgacccagccttaaacttactactctacacaggatgcctggccgagggagagctggaggtgacagatcagctcccagggcaaagcgaccaaccctgggagcaggccctgaaccgcttctgcgattacctgcgctgggtgcagacgctttctgaccaggtccaggaagagctgcagagctcccaagtcacacaggaactgacgtgagtgctcagcgcttcaccctccgcacctgctgagtatccagatccaggggttcctcctatctgggcacctacctacttgtttcctttctccatgagtgtgtgggccaggttggccttgaactctcaatacttctgctttctttagccttctggatactgggatgaacaggcattcatt

SEQ ID NO: 3 - DNA, 300 bp, Rattus rattus
aagaagaagaagaccccagcgaggaaaatgtgctggagacccctgtgccggttcctgtggctttggtcctatctgtcctatgttcaagctgtgcctatccacaaagtccaggatgacaccaaaaccctcatcaagaccattgtcaccaggatcaatgacatttcacacacggtagggagtctcttgtggggaagcaaaggagaagtagggctagaaccagagtctgagaatcacagtgagcgccttctgagtgtcatgcaccccctagaacctgagaatttgtaagcctagggtgtccat

SEQ ID NO: 4 - DNA, 28 bp, Rattus rattus
aagcggttcagggcctgctcccagggtt

SEQ ID NO: 5 - DNA, 28 bp, Rattus rattus
gggattacctgcgctgggtgcagacgct

SEQ ID NO: 6 - DNA, 28 bp, Rattus rattus
gtggataggcacagcttgaacataggac

SEQ ID NO: 7 - DNA, 28 bp, Rattus rattus
aagtccaggatgacaccaaaaccctcat

SEQ ID NO: 8 - DNA, 20 bp, Rattus rattus
ccccagtttgtggtctgcca

SEQ ID NO: 9 - DNA, 18 bp, Rattus rattus
gctaaaggtgaagatcta

SEQ ID NO: 10 - protein, 35 aa, Artificial Sequence
  SYNTHESIZED
RSDALSVDSSHRTRRSDNLSETSGSLTRRSDDLTR

SEQ ID NO: 11 - protein, 35 aa, Artificial Sequence
  SYNTHESIZED
RSDHLSRQSSDLRRRSDVLSADRSNRIKTSSNLSR

SEQ ID NO: 12 - protein, 28 aa, Artificial Sequence
  SYNTHESIZED
RSDALSEQNATRTKRSDYLSTQNAHRKT

SEQ ID NO: 13 - protein, 35 aa, Artificial Sequence
  SYNTHESIZED
DQSTLRNDRSNLSRTSANLSRRSDNLSEDRSALAR